(19) United States
(12) Patent Application Publication
(10) Pub. No.: US 2008/0248136 A1
(43) Pub. Date: Oct. 9, 2008

(54) ACUTE AND CHRONIC HEARTBURN COMPOSITION AND METHOD

(75) Inventors: Ken Clement, Mississauga (CA); Shan Chaudhuri, Mississauga (CA); Megan Thomas, Mississauga (CA)

Correspondence Address: TORYS LLP, 79 WELLINGTON STREET WEST, SUITE 3000, BOX 270, TD CENTRE, TORONTO, ON, M5K 1N2, CA

(73) Assignee: IOMEDIX DEVELOPMENT INTERNATIONAL SRL, St. Michael (BB)

(21) Appl. No.: 11/867,474

(22) Filed: Oct. 4, 2007

Related U.S. Application Data
(60) Provisional application No. 60/862,886, filed on Oct. 25, 2006.

(51) Int. Cl. A61K 33/08; A61K 33/10; A61P 1/04

(52) U.S. Cl. 424/687; 424/690; 424/692

(57) ABSTRACT

The present invention relates to a composition and method for the simultaneous alleviation of the symptoms form both acute and chronic gastric and esophageal reflux disorder. The composition of the present invention comprises at least calcium carbonate, aluminum hydroxide, or magnesium hydroxide and limonene, wherein the metal salts treat the acute case of the aforementioned disorder and the limonene is used to treat the chronic case.

### RELATED APPLICATIONS

The application is related to and claims benefit of priority to U.S. Provisional Patent Application Ser. No. 60/862,886 entitled “Acute and Chronic Heartburn Composition and Method,” filed Oct. 25, 2006, the disclosure of which is hereby fully incorporated by reference.

### FIELD OF THE INVENTION

The present invention relates to methods and compositions for alleviating heartburn. Specifically, the present invention relates to methods and compositions for alleviating the symptoms of acute and chronic gastric and esophageal reflux disorder.

### BACKGROUND

Gastric and esophageal reflux disorder, also known broadly as “heartburn”, occurs when the lower esophageal sphincter fails to close adequately after food has entered the stomach. As such, the contents of the stomach can then enter the lower portion of the esophagus. The gastric juices mixed with the stomach contents, which have a low pH as a result of hydrochloric acid content, can then irritate the esophageal wall, resulting in a burning sensation.

Antacids for the acute treatment of heartburn have been traditionally used. A blend of magnesium and aluminum hydroxide salts has been marketed for decades to provide a high-potency instantaneous treatment for heartburn. A salt, calcium carbonate, has also been used for this purpose. These salts are known to provide their effect on the acute alleviation of heartburn by exploiting their basic properties to neutralize gastric juices. However, the acute treatment of heartburn fails to address the needs of those who suffer from chronic and long term effects of heartburn and other gastrointestinal disorders.

Other methods and pharmacological treatments for heartburn or gastric and esophageal reflux disorder have been developed and marketed with commercial success. The histamine H2 receptor, which is predominantly found in the gastrointestinal tract, when activated stimulates the secretion of stomach acid, which contributes to heartburn and gastric reflux by increasing the amount of acid in the stomach. As such, antagonists to the H2 receptor have been developed and marketed for the alleviation and treatment of gastric and esophageal reflux disorder symptoms in chronic cases.

As an alternative to pharmacological treatments, U.S. Pat. No. 6,420,435 entitled “Method for Treating Gastrointestinal Disorders” purports to disclose a method of treating heartburn, gastric and esophageal reflux disorders, and gastric indigestion through the administration of therapeutically effective amounts of limonene. The patent discloses that daily or every other day administration of limonene reduced the severity of gastric and esophageal reflux disorder symptoms, as evaluated by subjects, to near complete relief of the symptoms after 14 days. Furthermore, the patent purports that in certain cases, the symptoms of gastric and esophageal reflux disorders were alleviated permanently. However, one striking disadvantage of U.S. Pat. No. 6,420,435 is that the use of limonene provides little, if any, immediate relief from the gastrointestinal disorder. Specifically, only 20% of all participants achieved complete relief of symptoms after 2 days of administration of the compound.

Conventionally, heartburn has been treated through acutely acting therapies addressing gastric pH, whereas chronic gastric and esophageal reflux disorders have been treated through chronic pharmacological intervention. In past approaches, acute and chronic treatments for this disorder have been addressed individually. Thus, it would be beneficial to provide both an alternative to pharmacological treatments for the disorders as well as treatment or alleviation of symptoms associated with gastric and esophageal reflux disorder at both the acute and chronic levels simultaneously.

### SUMMARY OF THE INVENTION

The foregoing needs and other needs and objectives that will become apparent for the following description are achieved in the present invention, for treating both acute and chronic gastric and esophageal reflux disorder. The present invention provides both a method and composition comprising an effective amount of limonene and at least one of calcium carbonate, aluminum hydroxide and magnesium hydroxide. The method provides orally administering to a mammal an effective amount of the composition.

### DETAILED DESCRIPTION OF THE INVENTION

In the following description, for the purposes of explanations, numerous specific details are set forth in order to provide a thorough understanding of the present invention. It will be apparent, however, to one skilled in the art that the present invention may be practiced without these specific details.

As used herein, “an effective amount” refers to an amount of a given substance or composition effective for providing acute and chronic alleviation of symptoms from gastric and esophageal reflux disorder when administered acutely or over a period of time in accordance with a predetermined dosage regimen.

As used herein, the term “nutritional composition” includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, “nutritional compositions” as disclosed herein belong to the category of compositions having at least one physiological function when administered to a mammal by conventional routes of administration.

As used herein the term “unmodified-release” format is understood to be defined as pertaining to the dissolution and bioavailability profile of an ingested dietary ingredient wherein no additional modifications, be it chemical or physical, have been made to the ingredient with the specific intent to alter the dissolution or bioavailability profile from that of ingredient in a naturally occurring form. It is also understood that unmodified-release is, essentially, immediate-release of active ingredients. This is further understood to be traditional- or conventional-release format where no slow-, delayed- or extended-release effect is incorporated.

As used herein the term “controlled-release” format is understood to be defined as a formulation of active ingredients and appropriate excipients in a specific format to facilitate a controlled- or non-immediate-release of active ingredients. The components of a controlled-release format may have been subjected to additional modifications, be it chemical or physical, with the specific intent to alter the dissolution or bioavailability profile from that of ingredient in a naturally occurring form.

As used herein the term “slow-release” format is understood to be defined as a controlled-release format wherein the release of active ingredients are delayed for a period of time or gradually released over an extended period of time. This is accomplished through the use of specific excipients and may include structural features designed to facilitate controlled-release. It is further understood that a slow-release format releases active ingredients at a rate slower than immediate-release.

As used herein the term “delayed-release” format is understood to be defined essentially as a controlled-release format wherein the components of the delayed-release format have undergone specific modifications, be it physical or chemical, to facilitate the release of active ingredients at a specific time after ingestion. It is further understood that delayed-release formats, release active ingredients at a period of time later than unmodified release.

As used herein the term “quick-release” format is understood to be defined essentially as ‘unmodified-release’, as defined above. However, the term “quick-release” may further include components having modifications, chemical or physical, to enhance the rate of dissolution or bioavailability of active ingredients.

The composition presented herein and those to be used in conjunction with methods presented herein are considered to be nutritional compositions useful for treating both acute and chronic gastric and esophageal reflux disorder as described in the present disclosure.

Alternatively, formulations and nutritional compositions belonging to the present invention may be considered to be nutraceuticals. As used herein, the term “nutraceutical” is recognized and used in the art to describe a specific chemical compound or combination of compounds found in, organic matter for example, which may prevent, ameliorate or otherwise confer benefits against an undesirable condition. As is known in the art, the term “nutraceutical” is used to refer to any substance that is a food, a part of food, or an extract of food which is suitable for consumption by an individual and providing physiological benefit which may be medical or health-related. Furthermore, the term has been used to refer to a product isolated, extracted or purified from foods or naturally-derived material suitable for consumption by an individual and usually sold in medicinal forms, such as caplets, tablets, capsules, soft gel capsules, soft-gel™ caplets, gel-caps and the like, not associated with food.

Embodiments of the present invention may employ particle-milling technology for enhanced utility and efficacy. U.S. patent application Ser. No. 11/709,526 entitled “Method For Increasing The Rate And Consistency Of Bioavailability Of Supplemental Dietary Ingredients” filed Feb. 21, 2007, herein fully incorporated by reference discloses the use of particle-milling for the purposes of increasing the rate of bioavailability following oral administration of components comprising supplemental dietary compositions. The increased bioavailability of a compound or ingredients is achieved via a reduction in particle size using a “fine-milling” technique. For the purposes of the present invention, the terms micronization, milling, particle-milling, and fine-milling are used interchangeably, wherein they refer to a technology, process and end-products involved in or leading to a narrowing of particle size range and a concomitant reduction in the average particle size. For the purposes of the present invention, acceptable milled-particle sizes are in the range of from about 1 nanometer to about 500 microns.

Further to improving bioavailability, it is understood by the inventors that increased solubility resulting from fine-milling will lead to improvements in characteristics in which solubility and reduced particle size likely play a role. The components of the present invention may be fine-milled in order to quicken the rate of dissolution.

Additionally, U.S. patent application Ser. No. 11/709,525 entitled “Method for a Supplemental Dietary Composition Having a Multi-Phase Dissolution Profile” filed Feb. 21, 2007, also fully incorporated by reference herein, discloses that components of the present invention may be used as portions of both non-milled and fine-milled, in order to provide a bi-phasic dissolution profile. Conventional oral dosage formulations are bound by the rate of dissolution of the unprocessed substance, thereby limiting the rate of bioavailability of the substance upon oral administration. This is particularly problematic for poorly-soluble compounds which have an inherently low rate of dissolution in that they may be excreted prior to first-pass.

It is herein understood that, due to the relationship between solubility and dissolution, the amount of a substance in solution at any given time is dependent upon both dissolution and solubility. Furthermore, it is understood by way of extension that increasing the rate of dissolution of a given substance acts to reduce the time to dissolution of a given solute or substance in a given solvent. However, the absolute solubility of said solute does not increase with infinite time. Thus, increasing the rate of dissolution of a substance will increase the amount of said substance in solution at earlier points in time, thus increasing the rate of bioavailability of said substance at earlier times upon oral administration.

The increase in the rate of bioavailability will allow better and quicker compound transfer to the systemic parts of the body.

Micronization is a technique which has been used as a method of sizing solid compounds to fine powders. Following a micronization process, compounds and more specifically poorly soluble compounds are transformed into fine powders which can then be transformed into suitable, stable and patient-compliant dosage forms. These forms, for the purposes of the present invention are derived for oral administration.

Micronization techniques offer an advantage over larger forms of compounds and poorly soluble compounds—following micronization, compounds have higher surface area to volume ratio. This provides for, as compared to physically coarse compounds, an ultrafine micronized powder that has a significantly increased total surface area. Mathematically, cross-sectional surface area increases with the square of the radius, while volume increases with the cube of the radius. Therefore, as a particle becomes smaller, the volume of the particle decreases at a faster rate than the surface area leading to an increase in the ratio of surface area to volume. By way of theoretical calculations, decreasing the size of a particle can increase its rate of dissolution via increasing the surface area to volume ratio. In the case of solubility, this increase in relative surface area allows for greater interaction with solvent. Further to such additional embodiments, components of the present invention may be present in portions fine-milled to varying degrees thereby providing a multi-phasic dissolution profile as is disclosed in the preceding application reference.

According to various embodiments of the present invention, the supplemental composition may be consumed in any form. For instance, the dosage form of the nutritional supplement may be provided as, e.g., a powder beverage mix, a liquid beverage, a dietary gel, a ready-to-eat bar or drink product. Preferably, acceptable oral dosage formats are provided as: a capsule, a liquid capsule, a caplet, a soft gel capsule, a soft-gel™ caplet, a tablet, a time-release capsule, a time-release liquid capsule, a time-release tablet, or as a time-release caplet. The preferred dosage form of the present invention is that of a soft-gel capsule.

It is herein understood that the components of the present invention are intended to be released and act in the stomach of an individual. As such, it is further understood that acceptable oral dosage forms preferably do not include chewable forms, which would release the components before contact with the stomach. Additionally, when presented in a chewable format the acidic saliva will tend to react with the antacids prior to entering the stomach, thereby rendering the antacids ineffective in reducing the symptoms of heartburn. As such, chewable formats of the present invention are not preferred.

The dosage form of the supplemental composition may be provided in accordance with customary processing techniques for herbal and nutritional supplements in any of the forms mentioned above. Additionally, the supplemental composition set forth in the example embodiment herein may contain any appropriate number and type of excipients, as is well known in the art.

The present invention is directed towards a method of treating both acute and chronic gastric and esophageal reflux disorder (heartburn) substantially simultaneously wherein the method comprises the oral administration of an effective amount of a composition comprising limonene and at least one of calcium carbonate, aluminum hydroxide and magnesium hydroxide to a mammal.

Antacids have been used to address the acute symptoms of several digestion-related disorders. For example, antacids have been used to treat duodenal and gastric ulcers, stress gastritis, gastric and esophageal reflux disorder, pancreatic insufficiency, biliary reflux, and constipation. Traditionally, calcium carbonate and other non-toxic metal salts known to neutralize an acidic environment have been employed. For example, aluminum hydroxide and magnesium hydroxide have been employed in treatments for the alleviation of acute gastric and esophageal reflux disorder symptoms. The group of Decktor showed that the administration of calcium carbonate, aluminum hydroxide or magnesium hydroxide one hour following a refluxogenic meal significantly increased esophageal pH. (Decktor D L, Robinson M, Maton P N, Lanza F L, Gottlieb S. Effects of Aluminum/Magnesium Hydroxide and Calcium Carbonate on Esophageal and Gastric pH in Subjects with Heartburn. Am J Ther. 1995 August; 2(8):546-552). A refluxogenic meal is one that is designed to induce acid reflux from the stomach into the esophagus, thereby inducing heartburn. Furthermore, the experiments of the Decktor et al. showed that aluminum hydroxide and magnesium hydroxide increased gastric pH above placebo levels, whereas, consistent with a calcium carbonate-induced “acid rebound” the calcium carbonate treated gastric pH remained at or below placebo levels.

In addition to the neutralization of gastric hydrochloric acid, a 1999 review article also discloses that the effect of traditional antacids is due partially due their ability to inhibit the proteolytic enzyme pepsin (Maton P N, Burton M E. Antacids revisited: a review of their clinical pharmacology and recommended therapeutic use. Drugs. 1999 June; 57(6):855-70). It has been shown that the proteolytic activity of pepsin falls rapidly at a pH of above 3 in gastric juice. Furthermore, it is understood that above this pH in vivo, all of the activity of pepsin is lost. Pepsin has been shown to increase five-fold in peptic ulcer disease to reach about 20% of the total activity in gastric juice, also having substantial mucolytic activity. As such, pepsin-induced mucosal damage can been shown to be related to high levels and activity of pepsin in gastric and esophageal reflux disorder (Allen A, Flemstrom G. Gastroduodenal mucus bicarbonate barrier: protection against acid and pepsin. Am J Physiol Cell Physiol. 2005 January; 288(1):C1-19. Review).

Moreover, the present invention provides a composition comprising limonene and at least one of calcium carbonate, aluminum hydroxide and magnesium hydroxide in amounts effective to alleviate the symptoms of gastric and esophageal reflux disorder.

It is understood by the inventors that calcium carbonate, aluminum hydroxide and magnesium hydroxide, being neutralizing compounds can aid in the alleviation of the symptoms of gastric and esophageal reflux disorder. This is achieved in a two-fold mechanism of acting as neutralizing agent in the low pH environment of gastric juice, and also by inhibiting the activity of pepsin, thereby inhibiting mucosal lining damage, resulting in decreased irritation of the mid and upper digestive system at high concentrations of released pepsin and high pepsin activity.

An embodiment of the present invention comprises between from about 100 mg to about 1500 mg of calcium carbonate acid per serving of the nutritional composition. In another embodiment, the nutritional composition comprises from about 100 mg to about 700 mg of calcium carbonate acid per serving of nutritional composition. In a further preferred embodiment, the nutritional composition comprises about 500 mg of calcium carbonate acid per serving of nutritional composition.

Another embodiment of the present invention comprises between from about 100 mg to about 1500 mg of aluminum hydroxide acid per serving of the nutritional composition. In an additional embodiment, the nutritional composition comprises from about 100 mg to about 700 mg of aluminum hydroxide acid per serving of the nutritional composition. In a further preferred embodiment, the nutritional composition comprises about 500 mg of aluminum hydroxide acid per serving of nutritional composition.

In another embodiment, the present invention comprises between from about 100 mg to about 1500 mg of magnesium hydroxide acid per serving of the nutritional composition. In an additional embodiment, the nutritional composition comprises from about 100 mg to about 700 mg of magnesium hydroxide acid per serving of the nutritional composition. In a further preferred embodiment, the nutritional composition comprises about 500 mg of magnesium hydroxide acid per serving of nutritional composition.

The present invention, as being designed to treat both acute and chronic gastric and esophageal reflux disorder symptoms, provides a component to treat the chronic aspects of the aforementioned disorder. The pH and pepsin activity, are in the case of the present invention attended to in the acute case of gastric and esophageal reflux disorder by the calcium carbonate, aluminum hydroxide and magnesium hydroxide. However the chronic aspects of the gastric and esophageal reflux disorder symptoms are attended to through administration of limonene.

Limonene, a hydrocarbon, belongs to the chemical class of terpenes and is a clear colorless liquid at room temperature. The rind of lemons and other citrus fruits contain considerable amounts of limonene, wherein it is mainly responsible for much of the citrus smell. As such, limonene is used as a flavoring agent in foods and added to products to lend a lemon or orange fragrance. Furthermore, as it is a chiral molecule biological sources produce the d-limonene enantiomer.

Unpublished clinical research studies have shown that the oral administration of 1000 mg of citrus peel extract standardized to 98.5% d-limonene every other day for 20 days can alleviate or reduce the symptoms of gastric and esphageal reflux disorder in individuals for period of six months or longer. Although no mechanism of action has been established at this time, it is understood that limonene confers its effects by providing a barrier on top of the gastric juice. It has also been postulated that the limonene may coat the esophagous and thus protect it against the caustic contents which would, when regurgitated from the stomach irritate the esophageal wall. Additionally, limonene may promote increased gastric emptying, thus riding the stomach of the gastric juices where they can no longer irritate the espophagous. In line with the notion of increased gastric emptying, the level of the contents contained within the stomach may be reduced faster, to a point wherein they are not as close to the lower esophageal sphincter, thus the stomach contents do not enter the esophagous and cause irritation since the refluxed material cannot reach this level.

Research in mice demonstrates that limonene has antinociceptive activity (do Amaral J F, Silva M I, de Aquino Neto M R, Neto P F, Moura B A, de Melo C T, de Araujo F L, de Sousa D P, de Vasconcelos P F, de Vasconcelos S M, de Sousa F C. Antinociceptive effect of the monoterpene R-(+)-limonene in mice. Biol Pharm Bull. 2007 July; 30(7):1217-20). This activity will act to lessen the feelings of pain and discomfort associated with heartburn. Additionally, it is well known that the bacteria H. Pylori is strongly associated stomach ulcers. Stomach ulcers are also associated with chronic gastric and esphogeal reflux disorder. It has been postulated that since limonene is a terpene, it may confer antibacterial activity against the H. Pylori bacteria, thereby offering a reduction or alleviation of the chronic symptoms of gastric and esophageal reflux disorder. Furthermore, the main metabolite of limonene, perillic acid, (Chow H H, Salazar D, Hakim I A. Pharmacokinetics of perillic acid in humans after a single dose administration of a citrus preparation rich in d-limonene content. Cancer Epidemiol Biomarkers Prev. 2002 November; 11(11):1472-6) inhibits mitogen-activated protein kinase signaling, which is stimulated by H. Pylori and contributes to it's pathogenesis (Chen Y C, Wang Y, Li J Y, Xu W R, Zhang Y L. H pylori stimulates proliferation of gastric cancer cells through activating mitogen-activated protein kinase cascade. World J Gastroenterol. 2006 October 7;12(37):5972-7).

An embodiment of the present invention comprises between from about 100 mg to about 1500 mg of citrus peel extract standardized to 98.5% d-limonene per serving of the nutritional composition. In another embodiment, the nutritional composition comprises from about 500 mg to about 1200 mg of citrus peel extract standardized to 98.5% d-limonene per serving of the nutritional composition. In a further preferred embodiment, the nutritional composition comprises about 1000 mg of citrus peel extract standardized to 98.5% d-limonene acid per serving of the nutritional composition.

It is understood by the inventors that individuals who suffer from gastric and esphageal reflux disorder would benefit from, and there is long felt want for, a composition and method for the substantially simultaneous alleviation of both acute and chronic symptoms of the disorder. The present invention provides composition for the simultaneous delivery of compounds to alleviate the acute and chronic symptoms of gastric and esphageal reflux disorder. By way of oral administration of the composition, to a mammal, of the present invention, a method is provided for the alleviation of the acute and chronic symptoms of gastric and esphageal reflux disorder. Furthermore, the “acid rebound” effect, common to antacids, wherein the desired antacid-induced increase in gastric pH is countered by a subsequent increase in acid production by the stomach, will be conveniently addresses by the present invention.

Although the following examples illustrate the practice of the present invention in three of its various embodiments, the examples should not be construed as limiting the scope of the invention. Other embodiments will be apparent to one skilled in the art from consideration of the specification of the following example.

### Extensions and Alternatives

In the foregoing specification, the invention has been described with specific embodiments thereof; however, it will be evident that various modifications and changes may be made thereto without departing from the broader spirit and scope of the invention.

### EXAMPLES

### Example 1

By way of example, the present invention may be provided in an embodiment comprising about 0.541 g of calcium carbonate and about 1.0 g of citrus peel extract standardized to 98.5% d-limonene. As a method for acutely alleviating the symptoms of gastric and esophageal reflux disorder, the composition of the present invention may be administer to a mammal as required. Furthermore, to alleviate the chronic symptoms of gastric and esophageal reflux disorder the composition of the present may be orally administered to a mammal at least once every other day.

### Example 2

By way of example, the present invention may be provided in an embodiment comprising about 0.5 g of aluminum hydroxide and about 1.0 g of citrus peel extract standardized to 98.5% d-limonene. As a method for acutely alleviating the symptoms of gastric and esophageal reflux disorder, the composition of the present invention may be administer to a mammal as required. Furthermore, to alleviate the chronic symptoms of gastric and esophageal reflux disorder the composition of the present may be orally administered to mammal at least once every other day.

### Example 3

By way of example, the present invention may be provided in an embodiment comprising about 0.5 g of magnesium hydroxide and about 1.0 g of citrus peel extract standardized to 98.5% d-limonene. As a method for acutely alleviating the symptoms of gastric and esophageal reflux disorder, the composition of the present invention may be administer to a mammal as required. Furthermore, to alleviate the chronic symptoms of gastric and esophageal reflux disorder the composition of the present may be orally administered to a mammal at least once every other day.

What is claimed:

1. A composition for treating gastric and esophageal reflux disorder, said composition comprising:
from about 0.09 g to about 1.45 g of limonene; and
from about 0.1 g to about 1.5 g of at least one antacid selected from the group of calcium carbonate, aluminum hydroxide and magnesium hydroxide.

2. The composition of claim 1 wherein the antacid is calcium carbonate.

3. The composition of claim 2, wherein the amount of limonene is about 0.985 g and the amount of calcium carbonate is about 0.541 g.

4. The composition of claim 1, wherein the limonene and the antacid comprise a solid oral dosage form having a multi-phasic rate of dissolution.

5. The composition of claim 4 wherein said multi-phasic rate of dissolution comprises a first-phase and a second-phase; whereby said first-phase has a first rate of dissolution said second-phase has a second rate of dissolution whereby the multi-phasic rate of dissolution provides a time-release mechanism.

6. The composition of claim 1, wherein the limonene and the antacid are present in unmodified-release formats.

7. The composition of claim 1, wherein said composition is provided to a mammal in need thereof in an acceptable oral dosage format.

8. The composition of claim 7, wherein the acceptable oral dosage format is selected from the group consisting of tablets, caplets, capsules and soft-gel capsules.

9. The composition of claim 7, wherein the acceptable oral dosage format is provided in a form selected from the group consisting of unmodified release and time-release.

10. The composition of claim 7, wherein the oral dosage format consists of an unmodified release in combination with a time-release format.

11. The composition of claim 9, where in the time-release formats are selected from the group of controlled-release, slow-release, delayed-release and quick-release.

12. A method for improving gastric and esophageal reflux disorder, the method comprising orally administering to a mammal a composition comprising:
from about 0.09 g to about 1.45 g of limonene; and
from about 0.1 g to about 1.5 g of at least one antacid selected from the group of calcium carbonate, aluminum hydroxide and magnesium hydroxide.

13. The method of claim 12 wherein the administration of the composition treats and alleviates the symptoms of acute gastric and esophageal reflux disorder.

14. The method of claim 12 wherein the administration of the composition prevents the symptoms of chronic gastric and esophageal reflux disorder.

15. The method of claim 12 wherein the limonene and the antacid comprise a solid oral dosage form having a multi-phasic rate of dissolution.

16. The method of claim 15 wherein the multi-phasic rate of dissolution comprises a first-phase and a second-phase; whereby said first-phase has a first rate of dissolution said second-phase has a second rate of dissolution whereby a the multi-phasic rate of dissolution provides a tie-release mechanism.

17. The method of claim 12, wherein the limonene and the antacid are present in unmodified-release formats.

18. The method of claim 12 wherein said composition is provided to a mammal in need thereof in an acceptable oral dosage format.

19. The method of claim 18, wherein the acceptable oral dosage format is selected from the group consisting of tablets, caplets, capsules and soft-gel capsules.

20. The method of claim 19, wherein the acceptable oral dosage format is provided in a form selected from the group consisting of unmodified release and time-release.

21. The method of claim 12, wherein the oral dosage format consists of an unmodified release in combination with a time-release format.
